(19) United States
(12) Patent Application Publication
(10) Pub. No.: US 2022/0313171 A1
(43) Pub. Date: Oct. 6, 2022

(54) Imaging and Treatment Methods for Early Detection of Diseases

(71) Applicant: Ishtar Faisal Algburi, London (CA)

(72) Inventor: Ishtar Faisal Algburi, London (CA)

(21) Appl. No.: 17/221,742

(22) Filed: Apr. 2, 2021

(51) Int. Cl. A61B 5/00; A61B 5/055; G01R 33/48; A61K 49/10; A61K 45/06; A61K 33/00; A61K 31/517; A61K 31/353; A61K 49/04; G01R 33/485; G01R 33/28

(52) U.S. Cl. CPC A61B 5/7275; A61B 5/055; A61B 5/0042; A61B 5/4082; A61B 5/4088; A61B 5/4094; G01R 33/4812; A61K 49/10; A61K 45/06; A61K 33/00; A61K 31/517; A61K 31/353; A61K 49/0438; G01R 33/485; G01R 33/282

(57) ABSTRACT

This invention relates to disease detection by imaging and treatment of virus infection. Previously, there was no way to use CEST MRI imaging to early detect and map the neurodegenerative diseases, multiple sclerosis disease, concussion, and traumatic brain injury. Also, previously, there was no way to use Computed Tomography (CT) imaging to early detect and map the neurodegenerative diseases. Embodiments of the present invention use a non-invasive CEST MRI imaging method is disclosed for early detection of diseases by using MRI or by using CT. The endogenous (MRI) contrast of the biological tissue can rely on the endogenous protons of the proteins and peptides as a source of the contrast, such as hydroxyl, amine, and amide protons, and thereby provide imaging and mapping for the early detection of the neurodegenerative diseases, multiple sclerosis disease, concussion, traumatic brain injury, and other diseases by using endogenous protons contrast via CEST MRI. Also, the exogenous agents can be used to produce MRI contrast, such as agents contain exchangeable protons and thereby provide imaging and mapping the inflammation in cancer and the expressed proteins in cancer cells for cancer detection. Also, using exogenous CT contrast agents for detection of amyloid beta, tau protein, alpha-synuclein protein, and aggregation proteins in neurodegenerative diseases and inflammation in many diseases such as neurodegenerative diseases, cancer and other inflammatory diseases Also, this invention relates to novel methods of treatment virus infection and enhance the immune system to produce antibodies against the viruses.

### INTRODUCTION

Embodiments of the invention described in this specification generally relate to disease detection by imaging, and more particularly, to novel imaging and treatment methods for early detection of disease by using chemical exchange saturation transfer (“CEST”) magnetic resonance imaging (“MRI”).

Positron emission tomography (PET) scanners are used for detection and mapping amyloid beta plaques, and neurofibrillary tangles in the brains of living people. PET scanners use radiotracers (“tracers”) and/or contrast agents to detect disease. When injected into the bloodstream of a patient, tracers or contrast agents cross quickly into the brain, where they bind to amyloid plaques or neurofibrillary tangles to mark them with emissions of mild radioactivity. Amyloid beta imaging is highly useful to enable people to begin therapy early enough in time to avoid or to significantly delay the development of neurodegenerative diseases. Amyloid beta proteins are aggregated and accumulated at the extracellular space and form large accumulations of aggregation proteins, called amyloid beta plaques, which cause neuronal death. In the progression of the disease, amyloid beta plaques precede tau tangles, and both cause eventual neural loss, the accumulation of amyloid in the brain has been identified as an early biomarker of some diseases, such as Alzheimer's disease. Also, MRI can be used to detect Alzheimer's disease but at a late stage where the structure of the brain had changed and enlarged the ventricles, also after atrophy the brain structures. There is no current method used for early detection of neurodegenerative diseases by using MRI before 10-20 years of the symptoms appear. The method presented in this invention can detect neurodegenerative diseases by using CEST MRI before 10-20 years of the symptoms appear by using endogenous contrast, which can be enhanced by glucose with or without monocarboxylic transporters (MCTs) inhibitor.

Multiple sclerosis (MS) is a disabling disease of the brain and central nervous system. Magnetic resonance imaging (MRI) can diagnose MS and monitor disease progression by using T₁-weighted brain MRI, but at a late stage, MRI cannot detect MS at an early stage. Some people with clinically-definite MS do not initially show lesions on MRI at the time of diagnosis. Also, MRI uses a contrast agent or tracer to detect active inflammation; this scan will highlight the new lesions or lesions that are growing. Also, these MRI methods are unable to detect small lesions; MRI scans can detect damage in the central nervous system.

In most cases, it helps to use a contrast agent, such as gadolinium, to identify the lesions better. However, gadolinium is expensive and has many side effects on humans, especially to humans with kidney diseases, also, cannot detect the lesions early. The method presented in this invention can detect multiple sclerosis (MS) by using CEST MRI before 10-20 years of the symptoms appear by using endogenous contrast which can be enhanced by administration glucose with or without monocarboxylic transporters (MCTs) inhibitor such as drugs like quercetin, atorvastatin, simvastatin, and other MCTs inhibitors.

A concussion is a mild traumatic brain injury (TBI) that results in a temporary loss of normal brain function. In many concussions, there are no external signs of head trauma. The most common cause of a concussion is a hard, direct hit to the head in contact sports such as football, soccer, and hockey. Repeated concussions can cause many problems, such as lasting cognitive. The current imaging for concussion using MRI or CT scans to make sure there is no bruising or bleeding in the brain, but these methods cannot detect the regions or the tissue under concussion or traumatic brain injury (TBI), they cannot map and the severity of the injury. The method presented in this invention can detect and map the tissue under concussion and traumatic brain injury, also, the method in this invention can monitor the recovery from concussion and traumatic brain injury very precisely by using endogenous contrast which can be enhanced by administration glucose with or without monocarboxylic transporters (MCTs) inhibitor.

Method of imaging the inflammation in cancer and the agents bind to expressed proteins in cancer cells for cancer detection by the administration of drugs or agents like polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others; flavonoids such as luteolin, quercetin, rutin, taxifolin, resveratrol, myricetin, rhein, and others; congo red and it's analogs such as chrysamine-g; nordihydroguaiaretic acid; tannins from brown algae such as phlorotannins include eckol, and it's derivatives and most polyphenols of seaweed; cannabinoids such as tetrahydrocannabinol (THC), cannabidiol (CBD), cannabigerol (CBG), and other cannabinoids; and/or administration of compounds contain at least phenol group with exchangeable one proton or more which can be detected by CEST-MRI and have ability to bind to human protein such as human serum albumin (HAS).

Antiviral drug resistance is a rising concern in immunocompromised patient populations. Rapid diagnosis of virus resistance can be made by incorporating viral mutations with resistance to various treatment of antiviral drugs. For example, when an influenza virus changes in the active site where an antiviral drug works, the influenza virus shows reduced susceptibility to that antiviral drug, which could be a sign of potential antiviral drug resistance. These viruses, such as Severe Acute Respiratory Syndrome coronavirus 2 (SARS-CoV2), Coronavirus disease (COVID-19), Middle East Respiratory Syndrome (MERS), influenza virus, Ebola virus, and Zika virus, and most other viruses can show reduced susceptibility to one or more antiviral drugs. Currently, no effective way to reduce the resistance of viruses to the antiviral drugs and/or methods to decrease the virus infection and reduce binding of viruses to the host cells and/or method to prevent viruses replication and/or methods of enhancing the immune system to kill the viruses by reducing the viruses replication and produce antibody against these viruses for future infection. The methods presented in this invention will induce acute acidification at the intracellular level to prevent virus replication and binding of the virus to the host cells. Also, this method will enhance the immune system to kill the viruses, prevent fusion of the virus to the cell, prevent virus protein RNA from replication in host cells and activate multiple mechanisms to kill the viruses inside and outside the host cells, these methods can apply on all the respiratory viruses and also, most other viruses.

Therefore, what is needed is a way to use CT to detect amyloid beta, tau protein, alpha-synuclein protein, and aggregation proteins in neurodegenerative diseases and inflammation in many diseases such as neurodegenerative diseases, cancer and other inflammatory diseases by binding and accumulation of the CT contrast agents to these proteins and diseases.

Also, a way to use CEST MRI imaging to detect and map neurodegenerative diseases, multiple sclerosis (MS), concussion, the inflammation in cancer, the agents bind to expressed proteins in cancer cells, and traumatic brain injury (TBI) by using endogenous and exogenous protons of tissue proteins and agents to detect these diseases, and a way to very precisely by using endogenous and exogenous protons contrast by CEST MRI as opposed to PET scans which are longer in time, not as safe as CEST MRI, and provide lower resolution images than CEST MRI, and to do so without injection or administration any expensive and unsafe contrast agents or tracers.

### DESCRIPTION

Novel imaging and treatment methods for early detection of disease using CEST MRI are disclosed. The novel imaging and treatment methods for early detection of disease using CEST MRI include a non-invasive CEST MRI imaging method for early detection of disease and a non-invasive CEST MRI for early detection of neurodegenerative diseases such as Alzheimer's disease.

In some embodiments, the novel imaging and treatment methods for early detection of disease and includes a plurality of steps comprising (i) acquiring, by way of a magnetic resonance imaging (MRI) machine, a T₂-image as an anatomical image, (ii) acquiring, by way of the MRI machine, a CEST image at S_{outside} as a reference image, (iii) acquiring, by way of the MRI machine, a plurality of CEST images at S_{within}, (iv) normalizing signal intensities of the acquired CEST images (S_{outside} and S_{within}) to the reference image, (v) calculating a contrast difference in S (ΔS contrast=S_{outside}−S_{within} for each image in the plurality of CEST images at S_{within}), and (vi) detecting disease and mapping disease severity based on the calculated ΔS contrast. In some embodiments, the CEST MRI imaging method for early detection of disease measures a chemical shift of the reference image>10 ppm or <−10 ppm. For example, the CEST MRI imaging method for early detection of disease can use 11 ppm or 20 ppm downfield or use −11 ppm or −20 ppm upfield.

In some embodiments, the novel imaging and treatment methods for early detection of neurodegenerative diseases and mapping the severity of neurodegenerative diseases includes a plurality of steps comprising (i) acquiring, by way of an MRI machine, a T₂-image as an anatomical image of the brain, (ii) acquiring, by way of the MRI machine, a CEST reference image at S_{outside}=11 ppm, (iii) acquiring, by way of the MRI machine, a plurality of CEST images at S_{within}=3.5 ppm or 3.4 ppm, (iv) normalizing signal intensities of the acquired CEST images (S_{outside} and S_{within}) to the reference image at S_{outside}=11 ppm, (v) calculating a contrast difference in S (ΔS contrast=S_{outside}−S_{within} for each image in the plurality of CEST images at S_{within}), and (vi) detecting early neurodegenerative diseases and mapping the disease in the tissue of the brain based on the calculated ΔS contrast.

In some embodiments, the novel imaging and treatment methods for early detection of the disease includes a plurality of steps comprising (i) acquiring, by way of a magnetic resonance imaging (MRI) machine, a T₂-image as an anatomical image, (ii) acquiring, by way of the MRI machine, a plurality of CEST images (before injection the agents) at saturation frequencies start from −20 ppm to 20 ppm with apart of 0.25 ppm, start and end saturation frequencies depend on agent's possible protons saturation frequencies that need to be administrated, for example resveratrol can be start from −7 ppm to 7 ppm with apart higher than or less than of 0.25 ppm, therefore acquiring images for the brain before injection of resveratrol from −7 ppm to 7 ppm with apart 0.25 ppm, (iii) acquiring, by way of the MRI machine, a plurality of CEST images (after injection the agents) at saturation frequencies start from −20 ppm to 20 ppm with apart higher than or less than of 0.25 ppm, start and end saturation frequencies depend on agent's possible saturation frequencies that need to be administrated, for example resveratrol can be start from −7 ppm to 7 ppm with apart higher than or less than 0.25 ppm, therefore acquiring images for the brain after administration of resveratrol from −7 ppm to 7 ppm with apart 0.25 ppm, using the saturation power and duration depend on the agents need to be administrated, if the agents contain exchangeable (hydroxyl protons and/or amine protons), these agents can be saturated by high power and short duration such as using 4.8 μT or less and 1 sec saturation time. Agents contain amid protons need to be saturated by lower power and short duration, such as using 1.5 μT and 4-5 sec saturation time.

(iv) (MTR) asymmetry before administration of the agents, acquiring CEST images at specific chemical shifts and at frequencies that decrease the magnetization of the exchangeable protons (M) of the administrated agents and also acquiring CEST images at specific chemical shifts at the opposite side of the same frequency (−Δω) which is decrease the magnetization of the exchangeable protons of the administrated agents at a frequency (−Δω) according to MTR_{asym-pre} (Δω)=Sₛₐₜ (−Δω)/Sₒ−Sₛₐₜ (Δω)/Sₒ, where Sₛₐₜ and Sₒ are the signal intensity obtained with and without selective saturation, respectively, for example, for resveratrol administration acquire images for the brain of mammalian at 7 ppm and −7 ppm before administration of resveratrol.

(v) (MTR) asymmetry after administration of the agents, such as administration of the agents by injections or orally to the animals or humans (mammalians), acquiring CEST images at specific chemical shifts and at frequencies that decrease the magnetization of the exchangeable protons (Δω)) of the administrated agents and also acquiring CEST images at specific chemical shifts at the opposite side of the same frequency (−Δω) which is decrease the magnetization of the exchangeable protons of the administrated agents at a frequency (−Δω) according to MTR_{asym-post} (Δω))=Sₛₐₜ (−Δω)/Sₒ−Sₛₐₜ (Δω)/Sₒ, where Sₛₐₜ and Sₒ are the signal intensity obtained with and without selective saturation, respectively, for example, for resveratrol administration acquire images for the brain of mammalian at 7 ppm and −7 ppm after administration of resveratrol.

(vi) calculating the difference in MTR_{asym} as MTR_{asym-contrast} (Δω)=MTR_{asym-post} (Δω)−MTR_{asym-pre} (Δω).

(vii) detecting disease and mapping disease severity based on the calculated MTR_{asym-contrast} (Δω), as the MTR_{asym-contrast} (Δω) increase as the disease is an increase in severity.

In some embodiments of this invention, (Δω) of the administrated agents can be calculated from phantoms mixed these agents with solution contain water and measure the (Δω) that have best peaks in the phantoms by MTR_{asym} spectrum (MTR_{asym} against frequencies (Δω)).

Method of imaging and detection of the inflammation in cancer and detection of aggressive cancer by agents, drugs, natural compounds, and any other chemical compounds accumulated and bind to expressed proteins in cancer cells and the inflammatory tissues by using CEST-MRI for agents contain exchangeable protons such as hydroxyl, amine, and amide protons, and thereby provide imaging and mapping for detection of the agents bind to expressed proteins in cancer cells and/or the inflammation in cancer.

Method of imaging the inflammation in cancer and the agents bind to expressed proteins in cancer cells by administration of drugs or agents like polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others; flavonoids such as luteolin, quercetin, rutin, taxifolin, resveratrol, myricetin, rhein, and others; congo red and it's analogs such as chrysamine-g; nordihydroguaiaretic acid; tannins from brown algae such as phlorotannins include eckol and it's derivatives and most polyphenols of seaweed; cannabinoids such as tetrahydrocannabinol (THC), cannabidiol (CBD), cannabigerol (CBG), and other cannabinoids; and/or administration of compounds contain at least phenol group with exchangeable one proton or more which can be detected by CEST-MRI and have ability to bind to human protein such as human serum albumin (HAS).

Novel imaging and treatment methods for early detection of diseases of the present disclosure may produce or result in the following elements. This list of possible constituent elements is intended to be exemplary only, and it is not intended that this list be used to limit novel imaging and treatment methods for early detection of diseases using CEST MRI of the present application to just these elements. Persons having ordinary skill in the art relevant to the present disclosure may understand there to be equivalent elements that may be substituted within the present disclosure without changing the essential function or operation of the novel imaging and treatment methods for early detection of diseases using CEST MRI.
Step 1. Acquiring anatomical T₂-image(s) of specific areas of the inflammation or the Tumor, which are the areas of focus as expected under the effects of certain diseases.
Step 2. CEST images (before administration of the agents)
Step 3. CEST images (after administration of the agents)
Step 4. Calculate (MTR) asymmetry before administration of the agents as
MTR_{asym-pre}.
Step 5. Calculate (MTR) asymmetry after administration of the agents as MTR_{asym-post}
Step 6. Calculating the difference in MTR_{asym} as MTR_{asym-contrast} (Δω)=MTR_{asym-post} (Δω)-MTR_{asym-pre} (Δω).
Step 7. Detecting disease and mapping disease severity based on the calculated MTR_{asym-contrast} (Δω), as the MTR_{asym-contrast} (Δω) increase as the disease is an increase in severity.

To use Novel imaging and treatment methods for early detection of disease for detection of diseases using CEST MRI of the present disclosure, a person working on MRI (an MRI operator) can follow the general steps noted above (Step 1, Step 2, Step 3, Step 4, Step 5, Step 6, and Step 7). Thus, to obtain accurate disease detection and severity mapped results, the MRI operator drives the actions noted in the steps of the methods by control operations with respect to the CEST MRI machine, which captures the images, and by interaction with a computing device, which performs computations, thereby allowing the MRI operator to follow the general steps (Step 1, Step 2, Step 3, Step 4, and Step 5, Step 6, and Step 7).

The MRI operator may carry out more specific steps. Furthermore, the MRI operator can produce MTR_{asym-pre}, MTR_{asym-post}, and MTR_{asym-contrast} by using MATLAB (MathWorks) on a computing device and thereby make determinations of diseases that may be present.

MTR_{asym-post} map and MTR_{asym-contrast} map can be used to detect the agents that bind to the inflammation in cancer and the expressed proteins in cancer cells, MTR_{asym-post} will be increased after administration of agents compared to MTR_{asym-pre}, which refer to increase binding of the agents to these proteins (the inflammation in cancer and the expressed proteins in cancer cells) as the disease is severe, the MTR_{asym-post} is significantly increased as the inflammation in cancer and the expressed proteins in cancer cells increased. Also, MTR_{asym-contrast} map can be used as a difference map to the detection of the agents bind to the inflammation in cancer, and the expressed proteins in cancer cells as MTR_{asym-contrast} is increased significantly as the inflammation in cancer and the expressed proteins in cancer cells increased.

MTR_{asym-post} map and MTR_{asym-contrast} map can be used to detect the agents that bind to the inflammation in cancer and the expressed proteins in cancer cells. MTR_{asym-post} will be increased after administration of agents compared to MTR_{asym-pre}, which refer to increase binding of the agents to the inflammation in cancer and the expressed proteins in cancer cells is severe, the MTR_{asym-post} is significant as increased in the inflammation in cancer and/or the expressed proteins in cancer cells. Also, MTR_{asym-contrast} map can be used as difference map to the detection of agents bind to the inflammation in cancer and/or the expressed proteins in cancer cells which can be detected by CEST-MRI and induce contrast after 10-15 mins to 2.5 hrs of administration these agents orally or by injections to animals or humans, MTR_{asym-contrast} is increased significantly as the inflammation in cancer and/or the expressed proteins in cancer cells is increased.

Also, the novel imaging and treatment methods for early detection of disease of the present disclosure are based on the use of CEST MRI. The endogenous magnetic resonance image (MRI) contrast of the biological tissue can rely on the endogenous protons of the proteins and peptides as a source of the contrast, such as hydroxyl, amine, and amide protons, and thereby provide imaging and mapping for the early detection of the neurodegenerative diseases, multiple sclerosis disease, concussion, and traumatic brain injury, by using endogenous protons contrast via CEST MRI, The difference in CEST images signals is used to early detect and map severity of the diseases and predict response to the treatment.

The novel imaging and treatment methods for early detection of disease using CEST MRI of the present disclosure may produce or result in the following elements. This list of possible constituent elements is intended to be exemplary only, and it is not intended that this list be used to limit the novel imaging and treatment methods for early detection of disease using CEST MRI of the present application to just these elements. Persons having ordinary skill in the art relevant to the present disclosure may understand there to be equivalent elements that may be substituted within the present disclosure without changing the essential function or operation of the non-invasive imaging and treatment methods for early detection of disease using CEST MRI.
1. CEST image-(the reference image at S_{outside})
2. CEST images (multiple images at S_{within})
3. The difference in magnetization (ΔS contrast images=S_{outside}−S_{within})
4. The difference in magnetization after administration of glucose with or without MCTs inhibitor (ΔSₚₒₛₜ contrast images=S_{outside post}−S_{within post})
5. Resulting in the detection of disease(s) and mapping of the severity of disease(s)

The novel imaging and treatment methods for early detection of disease using CEST MRI of the present disclosure generally work by a sequence of actions or operation (steps). While the non-invasive imaging and treatment methods for early detection of disease using CEST MRI are described further below, the methods are generally detailed as follows.

Step 1: Acquiring anatomical T₂-image(s) of specific areas of the brain, which are areas of focus as expected under the effects of certain particular diseases. For instance, specific areas of the brain include the hippocampus and the cortex in Alzheimer's disease.

Step 2: Acquiring a CEST image (reference image, S_{outside}) at a specific chemical shift and at a signal frequency outside the range of frequency that decreases magnetization of related proteins.

Step 3 (or contemporaneously with Step 2): Acquiring CEST images at specific chemical shifts and at frequencies that decrease the magnetization of the proteins (S_{within}).

Step 4: This step continues in case administration of glucose with or without MCTs inhibitor, acquiring a CEST image (reference image, S_{outside post}) at a specific chemical shift and at a signal frequency outside the range of frequency that decreases magnetization of related proteins.

Step 5: (or contemporaneously with Step 4) in case administration of glucose with or without MCT inhibitor, acquiring CEST images at specific chemical shifts and at frequencies that decrease the magnetization of the proteins (S_{within post})

The CEST chemical shift correction is preferred to be done when Bo variation is larger than 8%. Also, all the CEST images (via signals intensities) are normalized to the reference image. For example, acquiring CEST images around 3.5 ppm for amide proton (between 3.1 ppm to 4 ppm with a step size of 0.1 ppm) as S_{within} and S_{within post} images, and acquiring CEST images around 2.5 ppm for amine proton (between 1 ppm to 3 ppm with a step size of 0.1 ppm) as S_{within} and S_{within} post images.

The novel imaging and treatment methods for early detection of disease using CEST MRI of the present disclosure support most of the pulse sequences for CEST images. For example, one may use fast spin-echo (FSE) pulse sequence by using the following parameters: TR=3 s; TE=6.4 ms; FOV=212×190 mm²; matrix size=256×256; slice thickness=4.4 mm; turbo-spin-echo factor=45; and single slice acquisition. The RF saturation section includes a series of four block RF saturation pulses (200 ms duration each and 2μT amplitude) at 3Tesla for the human.

To use the novel imaging and treatment methods for early detection of disease using CEST MRI of the present disclosure, a person working on MRI (an MRI operator) can follow the general steps noted above (Step 1, Step 2, Step 3, Step 4, and Step 5). Thus, to obtain accurate disease detection and severity mapped results, the MRI operator drives the actions noted in the steps of the methods by control operations with respect to the CEST MRI machine, which captures the images, and by interaction with a computing device, which performs computations, thereby allowing the MRI operator to follow the general steps (Step 1, Step 2, Step 3, Step 4, and Step 5), the MRI operator will do Step 4 and Step 5 in case there is glucose with or without MCTs inhibitor to get the results.

The MRI operator may carry out more specific steps. Furthermore, the MRI operator can produce ΔS, and ΔSₚₒₛₜ images contrast by using MATLAB (MathWorks) on a computing device and thereby make determinations of diseases that may be present.

For instance, since ΔS significantly decreases for brain tissues such as cortex and hippocampus at early Alzheimer's disease as increase the lactate and the acid load at early Alzheimer's disease before the symptoms appear, ΔS can be used to early detection and identify the severity of neurodegenerative diseases that involve early metabolic change. The MRI operator can detect many such diseases and map the severity by adhering to the following observations and rules: ΔS contrast can detect and map the distribution of the neurodegenerative disease in the areas or the structures such as the hippocampus and the cortex that are susceptible to early metabolic change such as in Alzheimer's disease before 10-20 years of the symptoms appear, but for controls ΔS contrast will be small change. ΔS contrast can be enhanced by administration of glucose at early detection of neurodegenerative diseases, with or without administration of monocarboxylic transporters (MCTs) inhibitor, where ΔSₚₒₛₜ is images contrast post administration of glucose combined with or without MCTs inhibitor is decreased significantly with administration of glucose combined with or without MCTs inhibitor for early detection of neurodegenerative diseases compared to controls as follow:

operation of the novel imaging and treatment methods for early detection of disease using CEST MRI after administration of glucose with or without MCT inhibitors:
1. CEST image (the reference image at S_{outside post}) after administration of glucose with or without MCTs inhibitor.
2. CEST images (multiple images at S_{within post}) after administration of glucose with or without MCTs inhibitor.
3. The difference in magnetization (ΔSₚₒₛₜ contrast images after=S_{outside post}−S_{within post}) after administration of glucose with or without MCTs inhibitor.
4. Resulting in the detection of disease(s) and mapping of the severity of the disease(s).

Early detection of neurodegenerative disease in this invention for all ages less than 65 years, also late neurodegenerative disease, referred to ages approximately beyond 65 years.

ΔS contrast (ΔS contrast=S_{outside}−S_{within}) without administration any drug can be used to early detection of neurodegenerative disease, the inflammation, and the atrophy in neurodegenerative disease from ΔS contrast value, (ΔS contrast) is decreased in early neurodegenerative disease from (ΔS contrast) it is possible to map the severity of the brain tissues of neurodegenerative disease, the inflammation, and the atrophy in neurodegenerative disease. In controls (ΔS contrast) will be higher.

To enhance the contrast in early detection of neurodegenerative disease ΔS_{within final} contrast the absolute value is increased significantly for brain tissues of the patients in the early detection of neurodegenerative disease, but for the controls the absolute value (ΔS_{within final}) will be small.

ΔS_{within final}=S_{within post}−S_{within}

Also, ΔS_{within final} the absolute value can be used to detect the area under inflammation in neurodegenerative diseases early; the ΔS_{within final} the absolute value is increased when the inflammation is increased.

At the late stage of neurodegenerative diseases beyond 65 years of age, ΔS_{within final} is small for late neurodegenerative disease patients, but ΔS_{within final} will be increased significantly for the brain tissue of the late controls (beyond 65 years age) due to continuing controls acid load.

Also, early detection of neurodegenerative diseases can be achieved by magnetic resonance spectroscopy (MRS) by increasing the signal of lactate at early neurodegenerative disorders compared to controls. Also, this signal can be enhanced by the administration of glucose with or without monocarboxylic transporters (MCTs) inhibitor such as drugs like quercetin, atorvastatin, simvastatin, and other MCT inhibitors. The signal comes from lactate will be higher in early detection of neurodegenerative diseases compared to small signal comes from lactate in controls.

Early detection of multiple sclerosis disease (MS) can be achieved by ΔS contrast (ΔS contrast=S_{outside}−S_{within}) without administration any drug can be used to early detection of multiple sclerosis, the inflammation, and the atrophy in multiple sclerosis from ΔS contrast value, (ΔS contrast) is decreased in early multiple sclerosis from (ΔS contrast) it is possible to map severity of the brain tissues of multiple sclerosis to detect the lesions and the inflammation, and the atrophy. In controls (ΔS contrast) will be higher.

To enhance the contrast in early detection of multiple sclerosis ΔS_{within final} contrast is increased significantly for brain tissues of the patients in the early detection of multiple sclerosis, the inflammation, and the atrophy in multiple sclerosis but the controls (ΔS_{within final}) will be very small.

ΔS_{within final}=S_{within post}−S_{within}

Also, ΔS_{within final} can be used to detect the area under inflammation in many diseases early; the ΔS_{within final} is increased when the inflammation is increased.

Also, early detection of multiple sclerosis can be achieved by magnetic resonance spectroscopy (MRS) by increasing the signal of lactate at early multiple sclerosis compared to controls; also this signal can be enhanced by administration of glucose with or without monocarboxylic transporters (MCTs) inhibitor, which the signal comes from lactate will be higher in early detection of multiple sclerosis and the inflammation area compared to small signal comes from lactate in controls.

Early detection of concussion and traumatic brain injury can be achieved by ΔS contrast (ΔS contrast=S_{outside}−S_{within}) without administration any drug can be used to early detection of concussion and traumatic brain injury, and the inflammation in concussion and traumatic brain injury from ΔS contrast value, (ΔS contrast) is decreased in early concussion and traumatic brain injury from (ΔS contrast) it is possible to map the severity of the brain tissues of concussion and traumatic brain injury to detect the tissue under the risk of acid load and the inflammation, ΔS contrast can be used to monitor the treatment of concussion and traumatic brain injury. In controls (ΔS contrast) will be higher.

To enhance the contrast in early detection of concussion and traumatic brain injury ΔS within final contrast is increased significantly for brain tissues of the patients in the early detection of the concussion, traumatic brain injury and the inflammation, ΔS within final can be used to monitor the treatment of concussion and traumatic brain injury. However, for the controls (ΔS_{within final}) will be very small.

ΔS_{within final}=S_{within post}−S_{within}

Also, ΔS_{within final} can be used to early to detect the area under inflammation in many diseases; the ΔS_{within final} is increased when the inflammation is increased.

Also, early detection of concussion and traumatic brain injury can be achieved by magnetic resonance spectroscopy (MRS) by increasing the signal of lactate at early concussion and traumatic brain injury compared to controls; also this signal can be enhanced by administration of glucose with or without monocarboxylic transporters (MCTs) inhibitor, which the signal comes from lactate will be higher in early detection of concussion and traumatic brain injury and the inflammation area compared to small signal comes from lactate in controls. Also, MRS can be used to monitor the treatment of concussion and traumatic brain injury.

The method presented in this invention is using Computed Tomography (CT) contrast agents to detect amyloid beta, tau protein, alpha-synuclein protein, and aggregation proteins in neurodegenerative diseases and inflammation in many diseases such as neurodegenerative diseases, cancer and other inflammatory diseases by binding and accumulation of the CT contrast agents to these proteins and diseases. CT contrast agents or compounds have a similar structure to the CT contrast agents and have binding ability to human proteins such as binding to human serum albumin (HSA) can be used as CT contrast agents to detect amyloid beta, tau protein, alpha-synuclein protein, and aggregation proteins in neurodegenerative diseases and inflammation in many diseases such as neurodegenerative diseases, cancer, and other inflammatory diseases. The protocol of using these contrast agents is similar to the CT protocol of using these contrast agents in regular CT scans for detection the other diseases such as the similarity in doses and CT scan time. For example, CT contrast agents such as iohexol, iophenoxic acid, iopanoic acid, and other contrast agents can be binding to human protein such as human serum albumin also, can bind to amyloid beta, tau protein, alpha-synuclein protein, and aggregation proteins also, can be accumulated in the inflammatory tissue by multiple binding to the proteins, such as in Parkinson's disease (PD), epilepsy, ischemic stroke, and Alzheimer's disease (AD), Multiple sclerosis (MS), Huntington's disease (HD), Amyotrophic Lateral Sclerosis (ALS), cancer, concussion, traumatic brain injury, and other neurodegenerative diseases and other inflammatory diseases.

Antiviral drug resistance is a rising concern in immunocompromised patient populations. Rapid diagnosis of virus resistance can be made by incorporating viral mutations with resistance to various treatment of antiviral drugs. For example, when an influenza virus changes in the active site where an antiviral drug works, the influenza virus shows reduced susceptibility to that antiviral drug, which could be a sign of potential antiviral drug resistance. These viruses such as Coronavirus (Coronaviridae), Severe acute respiratory syndrome (SARS), Severe Acute Respiratory Syndrome coronavirus 2 (SARS-CoV2), Coronavirus disease (COVID-19), Middle East Respiratory Syndrome (MERS), influenza virus, Ebola virus, and Zika virus, human immunodeficiency virus (HIV), hepatitis C virus (HCV), oncoviruses, and most other viruses which are the hosts are humans and/or animals and/or insects can show reduced susceptibility to one or more antiviral drugs. Currently, no effective way to reduce the resistance of viruses to the antiviral drugs and/or methods to decrease the virus infection and reduce binding of viruses to the host cells and/or method to prevent viruses replication and/or methods of enhancing the immune system to kill the viruses by reducing the viruses replication and produce antibody against these viruses for future infection. The methods presented in this invention will induce acute acidification at the intracellular level to prevent virus replication in the infected cells and prevent binding of the virus to the host cells; also, this method will enhance the immune system to kill the viruses, prevent fusion of the virus to the cell, release the virus, prevent virus protein RNA from replication in host cells and activate multiple mechanisms to kill the viruses inside and outside the host cells, these methods can apply on all the respiratory viruses and also, most other viruses. Also, these treatments can prevent virus infection, enhance exist virus treatment by reducing the resistance of the virus to the antiviral drugs, enhance the immune system to kill the viruses; therefore, the body will produce antibodies for future viruses infection after killing the viruses in the body which could vaccinate the body against the future infection. It is possible with this treatment to give small doses of the viruses to the body combined with the treatment methods presented in this invention to activate the immune system to produce antibodies and activate other immune cells to develop the internal antibody against these viruses, which could vaccinate the body against the future infection.

Methods of treatment, prevention, and enhance the immune system to kill viruses include administration of monocarboxylate transporter (MCTs) inhibitors (such as, flavonoids such as luteolin, quercetin, and others; polyphenols such as curcumin, rosmarinic acid, epigallocatechin gallate, and others; cinnamate, a-cyano-4-hydroxycinnamate (4-CIN), lonidamine, diclofenac, syrosingopine, atorvastatin, simvastatin, and other analogs) combined with or without administration of glucose, also, combined with or without antiviral drugs such as: Angiotensin converting enzyme 2 inhibitor (ACE-2 inhibitor) such as some polyphenols (epigallocatechin gallate, and others), drugs anti RNA polymerase and synthesis such as favipiravir, drugs inhibit M2 protein (H⁺ pump) such as amantadine, drugs inhibit hemagglutinin such as arbidol (umifenovir), drugs inhibit virus release such as zanamivir and oseltamivir (neuraminidase (NA) inhibitors), drugs increase endosome and the lysosome pH such as chloroquine and hydroxychloroquine, antibiotic such as azithromycin, and other antiviral drugs) sometimes one administrated drug can target and inhibit two or more mechanisms in the viruses infected or host cells. The method of the treatment comprising administration of drugs to treat the viral infection, prevent the infection, and enhance the immune system to kill the viruses and enhance the immune system to kill the viruses, therefore, the body will produce antibodies for future viruses infection after killing the viruses in the body which could vaccinate the body against the future infection, these treatments include administration of drugs such as:

1. GLUT (glucose transporter) inhibitors such as phloretin, fasentin, quercetin, EGCG, and other drugs.
2. Na⁺/H⁺ exchange inhibitors, such as cariporide, EIPA, amiloride, or another active analog derivative of amiloride.
3. Pyruvate dehydrogenase kinase (PDK) inhibitors, such as Sodium Phenylbutyrate (4-PBA), sodium dichloroacetate, resveratrol, quercetin dihydrate, dicumarol, and other analogs.
4. Carbonic Anhydrases (CA) inhibitors, acetazolamide, methazolamide, and other analogs.
5. EGFR (epidermal growth factor receptor) proteins inhibitors like erlotinib, gefitinib, afatinib, cetuximab, necitumumab, and other analogs.
6. Increase extracellular pH (pHₑ) by administration agents contain bicarbonate such as sodium bicarbonate (NaHCO₃) and other agents.
7. HK (hexokinase) inhibitors like 3-bromopyruvate, 2-deoxyglucose, lonidamine, chrysin, resveratrol, and other analogs.
8. PKM2 (pyruvate kinase M2) inhibitors such as resveratrol, apigenin, and other drugs.
9. LDH-A (lactate dehydrogenase A) inhibitors like galloflavin and other drugs.
10. GLS (glutaminase) inhibitors such as CB-839, and BPTES (bis-2-(5-phenylacetamido-1,2,4-thiadiazol-2-yl) ethyl sulfide 3) and other drugs
11. Anti-inflammation drugs (NSAIDs) drugs like sodium salicylate, aspirin, diclofenac, celecoxib, and other drugs.
12. Mitochondria complex I inhibitors such as rotenone, metformin, phenformin, and other drugs.
13. Mitochondria complex II inhibitors, such as atpenins, HQNO, carboxin and TTFA, and other analogs.
14. Mitochondria complex III inhibitors, such as Antimycins, Myxothiazol, stigmatellin, strobilurins, Qo inhibitors, strobilurin, propylhexedrine, atovaquone, and other drugs.
15. Mitochondria complex IV inhibitors, such as sodium azide, cyanide, and other drugs.
16. Mitochondria complex V inhibitors (ATP synthesis inhibitors) such as pantoprazole, omeprazole, dexlansoprazole, esomeprazole, a bafilomycin, dorafem, rabeprazole, and it's analog and other drugs.
17. The proton pump inhibitors, comprises a vacuolar ATPase inhibitor (V-ATPase), such as pantoprazole, omeprazole, dexlansoprazole, esomeprazole, a bafilomycin, dorafem, rabeprazole, and other analogs.
18. The proton pump inhibitors V-ATPase comprises a H+/K+-ATPase inhibitors, such as pantoprazole, omeprazole, dexlansoprazole, esomeprazole, a bafilomycin, dorafem, rabeprazole, and other analogs.
19. The hyperthermia therapies

20. Chemotherapy administration that induces intracellular acidification, for example, cisplatin and it's analog and other chemotherapy that induce intracellular acidification.

1. The novel imaging and treatment methods for early detection of disease comprising:
acquiring, by way of a magnetic resonance imaging (MRI) machine, a T2-image as an anatomical image;
acquiring, by way of the MRI machine, a CEST reference image at a particular S_{outside} chemical shift and at a signal frequency outside a range of frequency that decreases magnetization of related proteins;
acquiring, by way of the MRI machine, a plurality of CEST images at a plurality of specific S_{within}, S_{within} post, S_{outside}, S_{outside} post, and chemical shifts and frequencies that decrease the magnetization of the related proteins;
calculating a difference in magnetization between S_{outside} and S_{within} at each specific S_{within} chemical shift and frequency for each image in the plurality of CEST images; and
detecting disease and mapping disease severity based on the calculated contrast differences before and after administration of glucose with or without monocarboxylic transporters (MCTs) inhibitor.

2. The novel imaging and treatment methods for early detection of disease of claim 1,
wherein detecting disease and mapping disease severity, comprises early detection of the neurodegenerative diseases.

3. The novel imaging and treatment methods for early detection of disease of claim 1, wherein the difference in magnetization contrast (ΔS contrast) is decreased in early neurodegenerative disease from (ΔS contrast) it is possible to map severity of the brain tissues of neurodegenerative disease, the inflammation, and the atrophy under and the inflammation in neurodegenerative disease. In controls (ΔS contrast) will be higher.

4. The novel imaging and treatment methods for early detection of disease of claim 1, wherein the difference in magnetization contrast (ΔS_{within final}) To enhance the contrast in early detection of neurodegenerative disease ΔS_{within final} contrast the absolute value is increased significantly for brain tissues of the patients in the early detection of neurodegenerative disease, but for the controls (ΔS_{within final}) the absolute value will be very small. (ΔS_{within final}) can be used to early detect the area under inflammation in neurodegenerative diseases, the ΔS_{within final} the absolute value is increased when the inflammation is increased.

5. The novel imaging and treatment methods for early detection of disease of claim 1, At the late stage of neurodegenerative diseases beyond 65 years age, ΔS_{within} final the absolute value is small for late neurodegenerative disease, but ΔS_{within} final the absolute value will be increased significantly for the brain tissue of the late controls beyond 65 years of age.

6. The novel imaging and treatment methods for early detection of disease, also, early detection of neurodegenerative diseases can be achieved by magnetic resonance spectroscopy (MRS) by increasing the signal of lactate at early neurodegenerative diseases compared to controls; also this signal can be enhanced by administration of glucose with or without monocarboxylic transporters (MCTs) inhibitor (such as, flavonoids such as luteolin, quercetin, and others; polyphenols such as curcumin, rosmarinic acid, epigallocatechin gallate, and others; cinnamate, a-cyano-4-hydroxycinnamate (4-CIN), lonidamine, diclofenac, syrosingopine, atorvastatin, simvastatin, and other MCT inhibitors). The signal comes from lactate will be higher in early detection of neurodegenerative diseases compared to small signal comes from lactate in controls. At the late stage of neurodegenerative diseases beyond 65 years of age, decrease the signal of lactate at late neurodegenerative diseases compared to controls in which the signal is higher.

7. The novel imaging and treatment methods for early detection of disease of claim 1, wherein detecting disease and mapping disease severity such as neurodegenerative diseases, multiple sclerosis disease, concussion, traumatic brain injury, and others, wherein the plurality of specific S_{within} chemical shifts decrease the magnetization from 10 ppm to 0.05 ppm downfield and −0.05 ppm to −10 ppm upfield.

8. The novel imaging and treatment methods for early detection of disease of claim 1, wherein detecting disease and mapping disease severity comprises multiple sclerosis (MS), ΔS contrast (ΔS contrast=S_{outside}−S_{within}) without administration any drug can be used to early detection of multiple sclerosis, concussion, traumatic brain injury, the inflammation, and the atrophy in multiple sclerosis, (ΔS contrast) is decreased in early multiple sclerosis, concussion, and traumatic brain injury from (ΔS contrast) it is possible to map severity of the brain tissues of multiple sclerosis, concussion, and traumatic brain injury to detect the lesions and the inflammation, and the atrophy. In controls (ΔS contrast) will be higher.

9. The novel imaging and treatment methods for early detection of disease of claim 8, to enhance the contrast in early detection of multiple sclerosis, concussion, and traumatic brain injury ΔS_{within} final contrast the absolute value is increased significantly for brain tissues of the patients in the early detection of multiple sclerosis, concussion, traumatic brain injury, the inflammation, and the atrophy in multiple sclerosis but the controls (ΔS_{within} final) the absolute value will be very small. Also, ΔS_{within} final the absolute value can be used to early detect the area under inflammation in many diseases; the ΔS_{within} final the absolute value is increased when the inflammation is increased.

10. The novel imaging and treatment methods for early detection of disease of claim 6, early detection of multiple sclerosis, concussion, and traumatic brain injury can be achieved by magnetic resonance spectroscopy (MRS) by increasing the signal of lactate at early multiple sclerosis, concussion, and traumatic brain injury compared to controls; also this signal can be enhanced by administration of glucose with or without monocarboxylic transporters (MCTs) inhibitor, which the signal comes from lactate will be higher in early detection of multiple sclerosis, concussion, traumatic brain injury and the inflammation area compared to small signal comes from lactate in controls.

11. The novel imaging and treatment methods for early detection of disease of claim 1, wherein endogenous protons of tissues can be used as an endogenous contrast to early detection of the multiple sclerosis disease, concussion, traumatic brain injury and the inflammation.

12. The novel imaging and treatment methods for early detection of disease comprising: imaging of agents bind to inflammatory tissues in cancer by decrease magnetization of the protons of these agents and transfer of these magnetizations to bulk water and surrounding tissues by way of a magnetic resonance imaging (MRI) machine, acquiring, by means of MRI machine, a T2-image as an anatomical image; obtaining, by means of the MRI machine, a plurality of CEST images (before injection the agents); acquiring, by means of the MRI machine, a plurality of CEST images (after administration of the agents); Calculate (MTR) asymmetry before administration of the agents MTRasym-pre; and Calculate (MTR) asymmetry after administration of the agents as MTRasym-post; and calculating the difference in MTRasym as
MTRasym-contrast (Δω)=MTRasym-post (Δω))−MTRasym-pre (Δω); detecting the accumulation of the agents (to enhance the CEST detection, it is possible to administration orally or by injection more than one agent) that can bind to inflammatory tissues and immune cells based on the calculated MTRasym-contrast (Δω), as the MTRasym-contrast (Δω) increase as the agents accumulated in the inflammatory tissues in cancer.

13. The novel imaging and treatment methods for early detection of disease of claim 12, comprising: administration of agents bind and accumulate in the inflammatory tissues and immune cells in cancer by using polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others; flavonoids such as luteolin, quercetin, rutin, taxifolin, resveratrol, myricetin, rhein, and others; congo red and it's analogs such as chrysamine-g; nordihydroguaiaretic acid; tannins from brown algae such as phlorotannins include eckol and it's derivatives and most polyphenols of seaweed; cannabinoids such as tetrahydrocannabinol (THC), cannabidiol (CBD), cannabigerol (CBG), and other cannabinoids; and/or administration of compounds contain at least phenol group with exchangeable one proton or more which can be detected by CEST-MRI and have ability to bind to human protein such as human serum albumin (HAS).

14. The novel imaging and treatment methods for early detection of disease comprising of claim 12: imaging of agents bind to the expressed proteins in cancer cells by decrease magnetization of the protons of these agents and transfer of these magnetizations to bulk water and surrounding tissues by way of a magnetic resonance imaging (MRI) machine, acquiring, by way of MRI machine, a T2-image as an anatomical image; acquiring, by way of the MRI machine, a plurality of CEST images (before injection the agents); acquiring, by way of the MRI machine, a plurality of CEST images (after administration of the agents); Calculate (MTR) asymmetry before administration of the agents MTRasym-pre; and Calculate (MTR) asymmetry after administration of the agents as MTRasym-post; and calculating the difference in MTRasym as
MTRasym-contrast (Δω)=MTRasym-post (Δω)−MTRasym-pre (Δω)); detecting the agents (to enhance the CEST detection, it is possible to administration orally or by injections more than one agent) that can bind to expressed proteins in cancer cells based on the calculated MTRasym-contrast (Δω), as the MTRasym-contrast (Δω) increase as the binding of agents to the target expressed proteins in cancer cells increase.

15. The novel imaging and treatment methods for early detection of disease of claim 14, comprising: administration of agents bind to expressed proteins in cancer cells orally, or by injection or administration multiple agents target and bind to the expressed proteins in cancer cells separately to enhance the detection of the expressed proteins in cancer cells in the same time which can be detected by CEST-MRI.

16. The novel imaging and treatment methods for early detection of disease of claim 14, comprising: administration of agents bind to expressed proteins in cancer cells orally or by injection by using polyphenols such as curcumin, rosmarinic acid, phenylindanc, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others; flavonoids such as luteolin, quercetin, rutin, taxifolin, resveratrol, myricetin, rhein, and others; congo red and it's analogs such as chrysamine-g; nordihydroguaiaretic acid; tannins from brown algae such as phlorotannins include eckol, and it's derivatives and most polyphenols of seaweed; cannabinoids such as tetrahydrocannabinol (THC), cannabidiol (CBD), cannabigerol (CBG), and other cannabinoids; and/or administration of compounds contain at least phenol group with exchangeable one proton or more which can be detected by CEST-MRI and have ability to bind to human protein such as human serum albumin (HAS), which can be detected by CEST-MRI.

17. The novel imaging and treatment methods for early detection of disease of claim 14, comprising: administration of agents bind to proteins involved in cell proliferation, survival, and angiogenesis in cancer cells.

18. The novel imaging and treatment methods for early detection of disease of claim 14, comprising: administration of agents bind to 67-kDa laminin receptor (67LR), and/or hypoxia-inducible factor (HIF-1α) and/or (HIF-1β), and/or epidermal growth factor receptor (EGFR), and/or antiapoptotic proteins such as Bcl-2 and Bcl-XL, and/or Glucose transporters 1-4 (GLUT1-4), and/or Peptidylprolyl Cis/Trans Isomerase, NIMA-Interacting 1 (PIN1), and/or vascular endothelial growth factor receptor (VEGFR), and/or insulin-like growth factor (IGFR), and/or DNA methyltransferase (DNMT), and other proteins which are elevated and expressed in cancer cells.

19. The novel imaging and treatment methods for early detection of the disease comprises, methods of treatment, prevention, and enhance the immune system to kill the viruses include administration of monocarboxylate transporter (MCTs) inhibitors combined with or without administration of glucose, also, combined with or without antiviral drugs, some drugs of below can target and inhibit two or more mechanisms in the viruses infected or hosted cells, the method of treatment comprising at least one of the list from (a) through (m):
(a). EGFR (epidermal growth factor receptor) proteins inhibitors like Afatinib and other drugs.
(b). Na+/H+ exchange inhibitors and/or Pyruvate dehydrogenase kinase (PDK) inhibitors.
(c). Chloride-bicarbonate (Cl/HCO3) exchange inhibitors and/or Carbonic Anhydrases (CA) inhibitors.
(d). GLUTs (glucose transporters) inhibitors and/or the proton pump inhibitor V-ATPase comprises a H+/K+-ATPase inhibitor.
(e). HK (hexokinase) inhibitors and/or PKM2 (pyruvate kinase M2) inhibitors.
(f). The hyperthermia therapies and/or Chemotherapy administration that induce intracellular acidification for example, cisplatin and it's analog and other chemotherapy that induce intracellular acidification.
(g). LDH-A (lactate dehydrogenase A) inhibitors and/or GLS (glutaminase) inhibitors.
(h). Anti-inflammation drugs (NSAIDs) and/or MD2M inhibitors.
(i). Mitochondria complex I inhibitors and/or mitochondria complex II inhibitors.
(j). Mitochondria complex III inhibitors and/or mitochondria complex IV inhibitors.
(k). The proton pump inhibitor, comprises a vacuolar ATPase inhibitor (V-ATPase) and/or mitochondria complex V inhibitors (ATP synthesis inhibitors).
(l). Increase extracellular pH (pHe) by administration agents contain bicarbonate such as sodium bicarbonate (NaHCO₃) and other agents.
(m). Drugs increase the endosome and lysosome permeability and cause them to leak, such as some polyphenols (epigallocatechin gallate and other drugs).
Wherein those of (a) through (m) that are in the formulation drugs are in amounts effective in a combination to treatment, prevention, and enhance the immune system to kill the viruses.

20. The formulation of claim 19, comprising at least two of (a) through (m).

21. The formulation of claim 19, comprising at least three of (a) through (m).

22. The formulation of claim 19, comprising at least four of (a) through (m).

23. The formulation of claim 19, comprising at least five of (a) through (m).

24. The formulation of claim 19, comprising at least six of (a) through (m).

25. The formulation of claim 19, comprising at least seven of (a) through (m).

26. The formulation of claim 19, comprising at least eight of (a) through (m).

27. The formulation of claim 19, comprising at least nine of (a) through (m).

28. The formulation of claim 19, comprising at least ten of (a) through (m).

29. The formulation of claim 19, comprising at least eleven of (a) through (m).

30. The formulation of claim 19, comprising at least twelve of (a) through (m).

31. The formulation of claim 19, comprising at least thirteen of (a) through (m).

32. The novel imaging and treatment methods for early detection of disease comprising: using CT contrast agents to the detection of amyloid beta in neurodegenerative diseases and other diseases by binding and accumulation of these contrast agent to the target proteins of amyloid beta by way of the CT machine; the CT images contrast (brightness of images) increase as the binding of these agents to the target amyloid beta and/or tau protein and/or protein aggregation and/or alpha-synuclein protein neurodegenerative diseases and other diseases increase.

33. The novel imaging and treatment methods for early detection of disease of claim 32, comprising: administration of CT contrast agents bind to amyloid beta proteins and tau proteins orally, or by injection or administration multiple agents target and bind to amyloid beta protein and tau proteins separately to enhance the detection of amyloid beta protein and tau protein in the same time which can be detected by CT machine.

34. The novel imaging and treatment methods for early detection of disease of claim 32, comprising: administration of CT contrast agents bind to amyloid beta proteins and alpha-synuclein protein orally or by injection or administration multiple agents target and bind to amyloid beta protein and alpha-synuclein protein separately to enhance the detection of amyloid beta protein and alpha-synuclein protein in the same time which can be detected by CT machine.

35. The novel imaging and treatment methods for early detection of disease of claim 32, comprising: administration of CT contrast agents bind to amyloid beta proteins and protein aggregation orally or by injection or administration multiple agents target and bind to amyloid beta protein and protein aggregation separately to enhance the detection of amyloid beta protein and protein aggregation in the same time which can be detected by CT machine.

36. The novel imaging and treatment methods for early detection of disease of claim 32, comprising: administration of CT contrast agents bind to amyloid beta protein, tau protein, alpha-synuclein protein, and protein aggregation orally or by injection or administration multiple agents target and bind to amyloid beta protein, tau protein, alpha-synuclein protein, and protein aggregation separately to enhance the detection of amyloid beta protein, tau protein, alpha-synuclein protein and protein aggregation in the same time which can be detected by CT machine.

37. The novel imaging and treatment methods for early detection of disease of claim 32, comprising: administration of CT contrast agents binds to amyloid beta proteins and/or tau protein and/or alpha-synuclein protein and/or protein aggregation such as iohexol, iophenoxic acid, iopanoic acid, and other contrast agents which can be binding to human protein such as human serum albumin.

38. The novel imaging and treatment methods for early detection of disease of claim 32, comprising: administration of CT contrast agents bind to amyloid beta proteins and/or tau proteins and alpha-synuclein protein orally or by injection or administration multiple agents target and bind to amyloid beta proteins and/or tau protein and alpha-synuclein protein separately to enhance the detection of amyloid beta proteins, tau protein and alpha-synuclein protein in the same time which can be detected by CT machine.

39. The novel imaging and treatment methods for early detection of disease of claim 32, comprising: administration of CT contrast agents bind to alpha-synuclein protein and/or tau proteins and proteins aggregation orally, or by injection or administration multiple agents target and bind to alpha-synuclein protein and/or tau protein and proteins aggregation separately to enhance the detection of alpha-synuclein protein, tau protein and proteins aggregation in the same time which can be detected by CT machine.

40. The novel imaging and treatment methods for early detection of disease comprising: using CT contrast agents to the detection of the inflammation at any part of the body and also, in the inflammatory diseases such as diseases of the skin, diseases of the nervous system, immune diseases, diseases of the musculoskeletal system, concussion, Alzheimer's disease, Parkinson disease, Huntington's disease, epilepsy, Down syndrome, Amyotrophic Lateral Sclerosis, Multiple sclerosis and other neurodegenerative diseases and other inflammatory diseases.

41. The novel imaging and treatment methods for early detection of disease of claim 40, comprising: administration of CT contrast agents bind to the inflammation tissue such as iohexol, iophenoxic acid, iopanoic acid, and other contrast agents which can be binding to human protein such as human serum albumin.

42. The novel imaging and treatment methods for early detection of disease of claim 40, comprising using CT contrast agents to the detection the inflammation in traumatic brain injury, ischemic stroke, and cancer by administration of CT contrast agents which can bind to the inflammation tissue such as iohexol, iophenoxic acid, iopanoic acid, and other contrast agents which can be binding to human protein such as human serum albumin.

43. The novel imaging and treatment methods for early detection of disease of claim 19, comprising: Treatment of viruses such as Coronavirus (Coronaviridae), Severe acute respiratory syndrome (SARS), Severe Acute Respiratory Syndrome coronavirus 2 (SARS-CoV2), Coronavirus disease (COVID-19), Middle East Respiratory Syndrome (MERS), influenza virus, Ebola virus, and Zika virus, human immunodeficiency virus (HIV), hepatitis C virus (HCV), oncoviruses, and all other respiratory, brain, liver, and immune deficiency viruses.

44. The novel imaging and treatment methods for early detection of disease of claim 19, comprising: Treatment of all other viruses which are the hosts are humans and/or animals and/or insects.
